(19) United States
(12) Patent Application Publication
(10) Pub. No.: US 2005/0209312 A1
(43) Pub. Date: Sep. 22, 2005

(54) Carboxylic acid derivatives of doxepin formulations preserved with low-irritation preservative

(75) Inventor: Glenn D. Smith, Rochester, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/082,269

(22) Filed: Mar. 17, 2005

Related U.S. Application Data
(60) Provisional application No. 60/554,811, filed on Mar. 19, 2004.

(51) Int. Cl. A61K 31/335

(52) U.S. Cl. 514/450

(57) ABSTRACT

The present invention relates to topical formulations used for treating allergic and inflammatory diseases. More particularly, the present invention relates to formulations of carboxylic acid derivatives of doxepin preserved with gentle preservative formulations and their use for treating and/or preventing allergic or inflammatory disorders of the eye and nose.

### CROSS-REFERENCE

This application claims the benefit of Provisional Patent Application No. 60/554,811 filed Mar. 19, 2004 and is incorporated herein by reference.

### FIELD OF THE INVENTION

The present invention relates to topical formulations used for treating allergic and inflammatory diseases. More particularly, the present invention relates to formulations of carboxylic acid derivatives of doxepin preserved with gentle preservative formulations and their use for treating and/or preventing allergic or inflammatory disorders of the eye and nose.

### DESCRIPTION OF THE RELATED ART

As taught in U.S. Pat. Nos. 4,871,865 and 4,923,892, both assigned to Burroughs Wellcome Co. (“the Burroughs Wellcome Patents”), certain carboxylic acid derivatives of doxepin, including olopatadine (chemical name: Z-11-(3-dimethylaminopropylidene)-6, 11-dihydrodibenz[b,e]oxepine-2-acetic acid), have antihistamine and antiasthmatic activity. These two patents classify the carboxylic acid derivatives of doxepin as mast cell stabilizers with antihistaminic action because they are believed to inhibit the release of autacoids (i.e., histamine, serotonin, and the like) from mast cells and to inhibit directly histamine's effects on target tissues. The Burroughs Wellcome Patents teach various pharmaceutical formulations containing the carboxylic acid derivatives of doxepin, including nasal spray and ophthalmic formulations. See, for example, Col. 7, lines 7-26, and Examples 8 (H) and 8 (I) of the '865 patent.

U.S. Pat. No. 5,116,863, assigned to Kyowa Hakko Kogyo Co., Ltd., (“the Kyowa patent”), teaches that acetic acid derivatives of doxepin and, in particular, olopatadine, have anti-allergic and anti-inflammatory activity.

Medicament forms taught by the Kyowa patent for the acetic acid derivatives of doxepin include a wide range of acceptable carriers; however, only oral and injection administration forms are mentioned.

U.S. Pat. No. 5,641,805, assigned to Alcon Laboratories, Inc. and Kyowa Hakko Kogyo Co., Ltd., teaches topical ophthalmic formulations containing olopatadine for treating allergic eye diseases. According to the '805 patent, the topical formulations may be solutions, suspensions or gels. The formulations contain olopatadine, an isotonic agent, and “if required, a preservative, a buffering agent, a stabilizer, a viscous vehicle and the like.” See Col. 6, lines 30-43. “[P]olyvinyl alcohol, polyvinylpyrrolidone, polyacrylic acid or the like” are mentioned as the viscous vehicle. See Col. 6, lines 55-57.

U.S. Pat. No. 5,736,165 teaches in-the-eye use of chlorine dioxide-containing formulations.

U.S. Patent Application Publication No. 20030055102 assigned to Alcon Research, Ltd. teaches topical formulations of olopatadine for treatment of allergic or inflammatory disorders of the eye and nose. According to the application, the aqueous formulations contain approximately 0.17-0.62% (w/v) of olopatadine and an amount of polyvinyl pyrolidone or polystyrene sulfonic acid sufficient to enhance the physical stability of the formulations. See Abstract.

PATANOL (olopatadine hydrochloride ophthalmic solution) 0.1% is currently the only commercially available olopatadine product for ophthalmic use. According to its labeling information, it contains olopatadine hydrochloride equivalent to 0.1% olopatadine, 0.01% benzalkonium chloride (BAK), and unspecified amounts of sodium chloride, dibasic sodium phosphate, hydrochloric acid and/or sodium hydroxide (to adjust pH) and purified water. It does not contain polyvinyl alcohol, polyvinylpyrrolidone, polyacrylic acid or any other polymeric ingredient. It has been recognized that the current ophthalmic formulation containing olopatadine demonstrates some irritation effect. This irritation effect is likely due to the use of BAK as a preservative.

In order for a drug to pass through the cornea, it must penetrate three layers of tissue, namely, the epithelium stroma and the endothelium. Except for highly lipophilic drugs, the epithelium is the main barrier to drug penetration of the cornea. Penetration of the stroma basically involves diffusion of the drug through a barrier that is approximately 360 microns thick. There are currently no known methods of enhancing drug penetration through the stroma or endothelium. However, it is possible to enhance the penetration of drugs through the epithelium, and thereby enhance the overall absorption of drugs applied topically to the eye.

Attempts to enhance the penetration of drugs through the corneal epithelium have included use of chemical agents to enhance the penetration of drugs through the epithelium. It has been reported that benzalkonium chloride (BAC), bile salts, dimethyl sulfoxide (DMSO), ethylenediamine tetraacetate (EDTA) and 1-dodecylazayl-cycloheptan-2-one (AZONE®) enhance the corneal penetration of certain drugs. The following publications may be referred to for further background concerning the use of such agents to enhance corneal penetration: Acta Ophthalmological, Vol. 53, p. 335 (1975); J. Pharm. Pharmacol., Vol. 39, p. 124 (1987); Chem. Abstracts, Vol. 106, 125931t, p. 402(1987); Journal of Pharmaceutical Sciences, Vol. 77, No. 1 (January, 1988); and Investigative Ophthalmology and Visual Science, Vol. 29, No. 2 (February, 1988).

Because some preservatives, such as BAK, are known to act as permeation enhancers, it would be expected that using a gentle preservative in place of BAK would undesirably affect the bioavailability and/or bioequivalence of active contained in the preserved formulation. A gentle preservative would be a preservative that when present in a preservative effective amount in the preserved formulation does not induce an undue amount of discomfort to a patient receiving a therapeutic dosage of the formulation.

Topical carboxylic acid derivatives of doxepin formulations that have prolonged therapeutic activity and are effective as products for treating allergic or inflammatory conditions in the eye and nose are desirable. Topical carboxylic acid derivatives of doxepin formulations that are effective as once-a-day products for treating allergic conditions in the eye are desirable. Topical carboxylic acid derivatives of doxepin formulations that are preserved with gentle preservatives while still maintaining desirable bioavailability and/or bioequivalence are also desirable.

### SUMMARY OF THE INVENTION

The present invention provides topical carboxylic acid derivatives of doxepin formulations that are effective for treating allergic or inflammatory disorders of the eye. The formulations of the present invention comprise approximately 0.01-5.0% of a carboxylic acid derivative of doxepin. In addition to the carboxylic acid derivative of doxepin, they also contain an amount of gentle preservative such as stabilized oxychloro complexes (otherwise known as PURITE) sufficient to preserve the formulations.

Among other factors, the present invention is based on the finding that gentle preservatives enhance the comfort of solutions containing approximately 0.01-1.0% carboxylic acid derivative of doxepin while still maintaining desirable bioequivalencei/bioavailability characteristics.

### DETAILED DESCRIPTION OF THE INVENTION

All references cited in this patent application are hereby incorporated herein by reference as if set forth in their entirety.

Carboxylic acid derivatives of doxepin are known compounds that can be obtained by the methods disclosed in U.S. Pat. No. 4,923,892. These compounds include:
1) (Z)-11-(3-(Dimethylamino)propylidene)-6,11-dihydrodibenz[b,e]oxepin-2-carboxylic acid
(2) (E)-11-(3-(Dimethylamino)propylidene)-6,11-dihydrodibenz[b,e]oxepin-2-carboxylic acid
(3) (E)-11-(3-(Dimethylamino)propylidene)-6,11-dihydrodibenz[b,e]oxepin-3-carboxylic acid
(4) (Z)-11-(3-(Dimethylamino)propylidene)-6,11-dihydrodibenz[b,e]oxepin-3-carboxylic acid
(5) (E)-11-(3-(Dimethylamino)propylidene)-6,11-dihydrodibenz[b,e]oxepin-8-carboxylic acid
(6) (Z)-11-(3-(Dimethylamino)propylidene)-6,11-dihydrodibenz[b,e]oxepin-8-carboxylic acid
(7) (E)-11-(3-(Dimethylamino)propylidene)-6,11-dihydrodibenz[b,e]oxepin-9-carboxylic acid
(8) (Z)-11-(3-(Dimethylamino)propylidene)-6,11-dihydrodibenz[b,e]oxepin-9-carboxylic acid
(9) (E)-11-(3-(Dimethylamino)propylidene)-6,11-dihydrodibenz[b,e]oxepin-2-acrylic acid
(10) (Z)-11-(3-(Dimethylamino)propylidene)-6,11-dihydrodibenz[b,e]oxepin-2-acrylic acid
(11) (E)-5-(3-(Dimethylamino)propylidene)-10,11-dihydro-5H-dibenzo[a,d]cyclohepten-3-carboxylic acid
(12) (Z)-5-(3-(Dimethylamino)propylidene)-10,11-dihydro-5H-dibenzo[a,d]cyclohepten-3-carboxylic acid

The carboxylic acid derivatives of doxepin useful in the present invention exist in either the cis (Z) or trans (E) isomers and mixtures of isomers. The formulations of the present invention can contain 0.01-5.0% of a carboxylic acid derivative of doxepin.

Generally, the carboxylic acid derivative of doxepin will be added in the form of a pharmaceutically acceptable salt. Examples of the pharmaceutically acceptable salts of carboxylic acid derivatives of doxepin include inorganic acid salts such as hydrochloride, hydrobromide, sulfate and phosphate; organic acid salts such as acetate, maleate, fumarate, tartrate and citrate; alkali metal salts such as sodium salt and potassium salt; alkaline earth metal salts such as magnesium salt and calcium salt; metal salts such as aluminum salt and zinc salt; and organic amine addition salts such as triethylamine addition salt (also known as tromethamine), morpholine addition salt and piperidine addition salt.

In addition to the carboxylic acid derivative of doxepin, the formulations of the present invention comprise suitable carriers and excipients as are known to those of skill in the art, for example, viscosity enhancing agents may be used to increase the viscosity of the formulations. Viscosity increased above that of simple aqueous solutions may be desirable to increase ocular absorption of the active compound, to decrease variability in dispensing the formulation, to decrease physical separation of components of a suspension or emulsion of the formulation and/or to otherwise improve the ophthalmic formulation. Such viscosity enhancing agents include as examples polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art. Povidone can be used as a suspending agent in the finished product and the water-soluble grades are routinely used in pharmaceuticals as a viscosity enhancing agent. The viscosity of aqueous solutions of the water-soluble grades of povidone depends on the average molecular weight. A subtle change in the grade and concentration of the viscosity enhancing agent can yield the desired characteristics. Povidone comes in a variety of grades, of which some are water soluble. Povidone K-90 is the highest molecular weight water-soluble viscosity grade Povidone. This material is listed as Povidone, USP 90,000. The high molecular grade povidone dissolves much more slowly than the lower molecular weight grade.

Ophthalmic formulations may also contain a surface-active agent that is acceptable for ophthalmic or otolaryngological uses. Frequently, this surfactant is non-ionic. Useful surface active agents include but are not limited to polysorbate 80, tyloxapol, TWEEN 80 (ICI America, Inc., Wilmington, Del.), PLURONIC F-68 (from BASF, Ludwigshafen, Germany) and the poloxamer surfactants and TETRONIC (from BASF, Ludwigshafen, Germany). These surfactants are nonionic alkaline oxide condensates of an organic compound that contains hydroxyl groups. The concentration in which the surface active agent may be used is only limited by neutralization of the bactericidal effects on the accompanying preservatives, or by concentrations that may cause irritation.

Ophthalmic formulations may also contain tonicity agents. The tonicity agents can be nonionic diols, preferably glycerol, in sufficient amounts to achieve isotonicity. The nonionic tonicity agents can be present in an amount of about 2 to 2.8% by weight, and preferably about 2.2 to 2.6%.

The formulations of the invention also may include additional therapeutic drugs such as drugs for treating glaucoma, anti-inflammatory drugs, anti-cancer drugs, anti-fungal drugs and anti-viral drugs. Examples of anti-glaucoma drugs include but are not limited to timolol-base, betaxalol, athenolol, levobanolol, epinenephrin, dipivalyl, oxonolol, acetazilumide-base and methazalomide. Examples of anti-inflammatory drugs include but are not limited to non-steroids such as piroxicam, indomethacin, naproxen, phenyylbutazone, ibuprofen and diclofenac.

The formulations of the present invention comprise about 0.01-1.0% carboxylic acid derivative of doxepin and relatively low concentrations of gentle preservative sufficient to provide an effectively preserved ophthalmic formulations. Formulations, which include, for example, such effective amounts of chlorine dioxide, have been found to be ophthalmically acceptable for in-the-eye use. The chlorine dioxide-containing formulations can be produced from precursor formulations including chlorine dioxide precursor components. Specific examples of chlorine dioxide precursor components include metal chlorites, such as alkali metal and alkaline earth metal chlorites. Technical grade sodium chlorite is a very useful chlorine dioxide precursor component. Chlorine dioxide-containing complexes, such as complexes of chlorine dioxide with carbonate, chlorine dioxide with bicarbonate and mixtures thereof are also included as chlorine dioxide precursor components. The exact chemical composition of many chloride dioxide precursor components, for example, stabilized chlorine dioxide (SCD) and the chlorine dioxide complexes, is not completely understood. The manufacture or production of certain chlorine dioxide precursor components is described in McNicholas U.S. Pat. No. 3,278,447. Specific examples of useful SCD products include that sold under the trademark DURA KLOR by Rio Linda Chemical Company, Inc., and that sold under the trademark ANTHIUM DIOXIDE by International Dioxide, Inc. An especially useful SCD is a product sold under the trademark PUROGENE by Bio-Cide International, Inc.

In general, the chlorine dioxide precursor component may be included in a medium, preferably a liquid medium, at a predetermined concentration, effective to produce the desired chlorine dioxide-containing composition.

Other gentle preservatives would include for example, saccharides such as are disclosed in U.S. patent application Ser. No. 10/725,159, filed Dec. 1, 2003, and U.S. application Ser. No. 10/725,049, filed Dec. 1, 2003.

The formulations of the present invention have a viscosity that insures that the product is comfortable, does not cause blurring, and is easily processed during manufacturing, transfer and filling operations.

In addition to the carboxylic acid derivative of doxepin and gentle preservative ingredients, the formulations of the present invention optionally comprise one or more excipients. Excipients commonly used in pharmaceutical formulations intended for topical application to the eyes or nose, such as solutions or sprays, include, but are not limited to, tonicity agents, preservatives, chelating agents, buffering agents, surfactants and antioxidants. Suitable tonicity-adjusting agents include mannitol, sodium chloride, glycerin, sorbitol and the like. Suitable chelating agents include sodium edetate and the like. Suitable buffering agents include phosphates, borates, citrates, acetates and the like. Suitable surfactants include ionic and nonionic surfactants, though nonionic surfactants are preferred, such as polysorbates, polyethoxylated castor oil derivatives and oxyethylated tertiary octylphenol formaldehyde polymer (tyloxapol). Suitable antioxidants include sulfites, ascorbates, BHA and BHT. The formulations of the present invention optionally comprise an additional active agent.

Particularly for formulations intended to be administered as eye drops, the formulations preferably contain a tonicity-adjusting agent in an amount sufficient to cause the final composition to have an ophthalmically acceptable osmolality (generally 150-450 mOsm, preferably 250-350 mOsm). The ophthalmic formulations of the present invention preferably have a pH of 4-8. Formulations of the present invention intended for use in the nose preferably have a pH of 3.5-8.

The formulations of the present invention can be packaged in opaque plastic containers. A suitable container for an ophthalmic product is a low-density polyethylene container that has been sterilized using ethylene oxide instead of gamma-irradiation. A suitable container for a nasal product is a high-density polyethylene container equipped with a nasal spray pump.

Without further elaboration, it is believed that one skilled in the art can, using the preceding description, utilize the present invention to its fullest extent.

1. A composition comprising a carboxylic acid derivative of doxepin and a gentle preservative.

2. The composition of claim 1 wherein the carboxylic acid derivative of doxepin is present in an amount of about 0.01 to about 5.0% (w/v).

3. The composition of claim 1 wherein the carboxylic acid derivative of doxepin is present in the form of a pharmaceutically acceptable salt.

4. The composition of claim 1 wherein the carboxylic acid derivative of doxepin is olopatadine.

5. The composition of claim 1 wherein the gentle preservative is a stabilized chlorine dioxide.

6. The composition of claim 1 wherein the gentle preservative is a preservative saccharide.

7. The composition of claim 1 further comprising a viscosity enhancing agent.

8. The composition of claim 7 wherein the viscosity enhancing agent is selected from the group consisting of s polyvinyl alcohol, polyvinyl pyrrolidone, methyl cellulose, hydroxy propyl methylcellulose, hydroxyethyl cellulose, carboxymethyl cellulose, hydroxy propyl cellulose and mixtures thereof.

9. The composition of claim 1 further comprising a surface-active agent.

10. The composition of claim 9 wherein the surface-active agent is a non-ionic surface-active agent.

11. The composition of claim 9 wherein the surface-active agent is selected from the group consisting of poloxamers, poloxamines, reverse poloxamers, reverse poloxamines and mixtures thereof.

12. The composition of claim 1 further comprising a tonicity agent.

13. The composition of claim 12 wherein the tonicity agent is selected from the group consisting of nonionic diols.

14. The composition of claim 5 wherein the stabilized chlorine dioxide is in the form of chlorine dioxide precursor components.

15. The composition of claim 14 wherein the chlorine dioxide precursor components are selected from the group consisting of metal chlorites, such as alkali metal and alkaline earth metal chlorites, chlorine dioxide-containing complexes, such as complexes of chlorine dioxide with carbonate, chlorine dioxide with bicarbonate and mixtures thereof.

16. The composition of claim 1 further comprising one or more excipients selected from the group consisting of chelating agents, buffering agents and antioxidants.

17. The composition of claim 12 wherein the tonicity agent is selected from the group consisting of mannitol, sodium chloride, glycerin, sorbitol and mixtures thereof.

18. The composition of claim 16 wherein the buffering agent is selected from the group consisting of phosphates, borates, citrates, acetates and the like.

19. The composition of claim 9 wherein the surface-active agent is selected from the group consisting of ionic and nonionic surfactants; polysorbates, polyethoxylated castor oil derivatives, oxyethylated tertiary octylphenol formaldehyde polymer and mixtures thereof.

20. The composition of claim 16 wherein the antioxidants is selected from the group consisting of sulfites, ascorbates, BHA, BHT and mixtures thereof.

21. The composition of claim 1 further comprising an additional active agent.

22. A method of inhibiting the release of histamine from a mast cell in a patient in need of treatment thereof comprising administering to the patient a composition comprising carboxylic acid derivative of doxepin and a gentle preservative.

23. The method of claim 22 wherein the carboxylic acid derivative of doxepin in the composition is present in an amount of about 0.01 to about 5.0 % (w/v).

24. The method of claim 22 wherein the carboxylic acid derivative of doxepin is present in the form of a pharmaceutically acceptable salt.

25. The method of claim 22 wherein the carboxylic acid derivative of doxepin is olopatadine.

26. The method of claim 22 wherein the gentle preservative is a stabilized chlorine dioxide.

27. The method of claim 22 wherein the gentle preservative is a preservative saccharide.

28. The method of claim 22 wherein the composition further comprises a viscosity enhancing agent.

29. The method of claim 28 wherein the viscosity enhancing agent is selected from the group consisting of polyvinyl alcohol, polyvinyl pyrrolidone, methyl cellulose, hydroxy propyl methylcellulose, hydroxyethyl cellulose, carboxymethyl cellulose, hydroxy propyl cellulose and mixtures thereof.

30. The method of claim 29 wherein the composition further comprises a surface-active agent.

31. The method of claim 30 wherein the surface-active agent is a non-ionic surface-active agent.

32. The method of claim 31 wherein the surface-active agent is selected from the group consisting of poloxamers, poloxamines, reverse poloxamers, reverse poloxamines and mixtures thereof.

33. The method of claim 22 wherein the composition further comprises a tonicity agent.

34. The method of claim 33 wherein the tonicity agent is selected from the group consisting of nonionic diols.

35. The method of claim 26 wherein the stabilized chlorine dioxide is in the form of chlorine dioxide precursor components.

36. The method of claim 35 wherein the chlorine dioxide precursor components are selected from the group consisting of metal chlorites, such as alkali metal and alkaline earth metal chlorites, chlorine dioxide-containing complexes, such as complexes of chlorine dioxide with carbonate, chlorine dioxide with bicarbonate and mixtures thereof.

37. The method of claim 22 wherein the composition further comprises one or more excipients selected from the group consisting of chelating agents, buffering agents and antioxidants.

38. The method of claim 33 wherein the tonicity agent is selected from the group consisting of mannitol, sodium chloride, glycerin, sorbitol and mixtures thereof.

39. The method of claim 37 wherein the buffering agent is selected from the group consisting of phosphates, borates, citrates, acetates and mixtures thereof.

40. The method of claim 30 wherein the surface-active agent is selected from the group consisting of ionic and nonionic surfactants such as polysorbates, polyethoxylated castor oil derivatives, oxyethylated tertiary octylphenol formaldehyde polymer and mixtures thereof.

41. The method of claim 37 wherein the antioxidants is selected from the group consisting of sulfites, ascorbates, BHA, BHT and mixtures thereof.

42. The method of claim 22 wherein the composition further comprises an additional active agent.

43. A drug product comprising the composition of claim 1 packaged in an opaque plastic container.

44. The drug product of claim 41 wherein the opaque plastic container is a low-density polyethylene container that has been sterilized.
